(19) United States
(12) Patent Application Publication
(10) Pub. No.: US 2022/0324843 A1
(43) Pub. Date: Oct. 13, 2022

(54) DICARBOXYLIC ACID BISAMIDE DERIVATIVES, USE THEREOF, PHARMACEUTICAL COMPOSITION BASED THEREON AND METHODS FOR PRODUCING DICARBOXYLIC ACID BISAMIDE DERIVATIVES

(71) Applicant: TREAMID THERAPEUTICS GMBH, BERLIN (DE)

(72) Inventors: Vladimir Evgenievich NEBOLSIN, Borzye (RU); Tatyana Alexandrovna KROMOVA, Kaluga (RU); Galina Alexandrovna ZHELTUKHINA, Moscow (RU)

(21) Appl. No.: 17/530,143

(22) Filed: Nov. 18, 2021

Related U.S. Application Data
(63) Continuation of application No. 14/879,648, filed on Oct. 9, 2015, now abandoned.
Application No. 14/879,648 is a continuation of application No. PCT/RU2014/000265, filed on Apr. 10, 2014.

(30) Foreign Application Priority Data
Apr. 12, 2013 (RU) 2013116822

(51) Int. Cl. C07D 403/12; C07D 233/64; C07D 235/14; C07D 277/64; C07D 277/28; C07D 417/12

(52) U.S. Cl. CPC C07D 403/12; C07D 233/64; C07D 235/14; C07D 277/64; C07D 277/28; C07D 417/12

(57) ABSTRACT

The present invention relates to novel biologically active compounds, in particular dicarboxylic acid bisamide derivatives of general formula I:

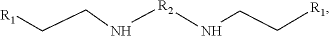

or pharmaceutically acceptable salts thereof, which are able to form complexes with or chelate metal ions. The invention also relates to the use of said compounds as an agent for the prevention and/or treatment of cardiovascular, viral, cancer, neurodegenerative and inflammatory diseases, diabetes, age-related diseases, diseases caused by microbial toxins, alcoholism and alcoholic cirrhosis, anaemia, porphyria cutanea tarda, and transition metal salt poisoning. The present invention also relates to novel methods for preparing dicarboxylic acid bisamide derivatives of general formula I.

### CROSS-REFERENCE

This application is a continuation of U.S. patent application Ser. No. 14/879,648, filed Oct. 9, 2015, which is a continuation of International Patent Application No. PCT/RU2014/000265, filed Apr. 10, 2014, which claims the benefit of priority from Russian Utility Patent Application No. 2013116822, filed on Apr. 12, 2013, the entire contents of each of which are hereby incorporated by reference.

The present invention relates to novel biologically active compounds, in particular, to dicarboxylic acid bisamide derivatives or pharmaceutically acceptable salts thereof, which are able to form complexes with, or chelate metal ions. The invention also relates to a use of said compounds as an agent for the prevention and/or treatment of cardiovascular, viral, oncological, neurodegenerative and inflammatory diseases, diabetes, age-related diseases, diseases caused by microbial toxins, alcoholism, alcoholic cirrhosis, anaemia, porphyria cutanea tarda, and transition metal salt poisoning.

### BACKGROUND

Metal ions are very important both in functioning of cells and the whole organism, and in the development of pathologies.

There are known drugs, the activity of which is determined by their ability to chelate metal ions. In this context, non-toxic compounds are currently being researched which are able to chelate metal ions with a high efficiency and selectivity and which are suitable for biomedical applications.

Compounds with chelating ability have been found among the various classes of compounds, such as mono- and dithiols, disulfides, azo compounds, nitrosoaromatic compounds, polyaminocarboxylic acid derivatives, thiosemicarbazones, pyridoxal isonicotinoyl hydrazone, quinoline, adamantane, pyrogallol, phenanthroline, thiopyrophosphates, etc. In addition, they have been found among other natural compounds such as, for example, carnosine, phytin and pectin. Compounds having several functional groups, which are able to act as electron donors in complex formation reactions, are of greatest interest. Thus, such compounds can act as ligands which specifically interact with metal ions or groups of metals.

Complex forming agents widely known today are polyaminocarboxylic acid derivatives (e.g., EDTA), D-penicillamine and polycyclic cryptands, which are successfully used in heavy metal poisoning therapy. In some iron-excessive conditions and hemochromatosis, deferoxamine is used as an iron chelator. In addition, chelators may be useful in therapy of pathologies associated with high calcium level conditions, for example, arthroses, atherosclerosis and renal lithiasis. It is also known that chelation therapy prevents cholesterol accumulation and restores its level in the blood, lowers the blood pressure, allows avoiding angioplasty, suppresses undesirable side effects of certain cardiac drugs, removes calcium from cholesterol plaques, dissolves thrombi and restores the blood vessel elasticity, normalizes arrhythmia, prevents aging, restores cardiac muscle force and improves cardiac function, increases the intracellular potassium concentration, regulates the mineral metabolism, is useful in the treatment of Alzheimer's disease, prevents cancer, improves the memory, and has a plurality of other positive effects. However, strong chelators currently used in chelation therapy have, in general, a toxic effect which manifests itself mainly in damage of the mucous membrane of the small intestine and in kidney dysfunction. In some cases, rapid administration of large amounts of known chelators can impair muscular irritability and blood clotting. Furthermore, strong chelators can interact with valuable bioelements (Na, K, Ca, Mg, and Ca) and can change the activity of vitally important metalloenzymes [Zelenin K. N. “Chelators in medicine,” Soros Educational Journal, 2001, v.7, No.1, pp. 45-50].

In this context, novel high-effective chelators are currently being researched which posess chelating ability that is sufficient for achieving a biological effect in vivo, and are free of side effects.

The idea of chelation therapy of viral diseases, such as HIV, human papilloma, herpes, hepatitis and the like, is especially actual.

Zinc-binding sites (so-called “zinc fingers”) in the structure of viral proteins are considered to be potential targets.

Today, several compounds have been found to affect the “zinc finger” structure of important proteins of these viruses.

The article of Andreas J. K., Bioorganic & Medicinal chemistry, 2003, v.11, pp. 4599-4613, discloses an adamantane derivative with trivial name Bananin which is a zinc-ion chelator. The compound is assumed to be chemically suitable for removing zinc from the HIV protein NCp7. Azodicarbonamide being an azoderivative that is supposed to exhibit the above-mentioned mechanism of action, is currently in phase I/II clinical trials against progressive AIDS.

The article of Rice W. G., Schaeffer C. A., Harten B., Nature, 1993, v.4, pp. 473-475, discloses 3-nitrozobenzamide which is able to remove zinc from the HIV protein NCp7 by inhibiting the replication of HIV and suppressing its pathogenicity in vitro and in vivo.

The “zinc finger”-like site of the human papilloma viral protein E6 was also selected as a target. This virus is a potential mediator in the etiology of cervical carcinoma.

The article of Beerheide W., Bernard H.-U., Tan Y.-J., Ganesan A. J., National Cancer Institute, 1999, v.91, No. 14, pp. 1211-1220, describes in vitro studies of aza compounds, and disulfide and nitrozo aromatic derivatives. 4,4-Dithiodimorpholine type compounds have been shown to provoke the release of zinc ions. As a result, a change is observed in the structure of the viral protein and impairment of its functions associated with the biology and pathology of human papilloma virus. However, clinical trials of said compounds are not yet completed, and their effectiveness can be judged only on the basis of in vitro studies.

The above-indicated compounds are assumed to be potential targets for the development of medicaments against cervical cancer, candyloma acuminate, and latent genital papillomavirus infections. Hepatitis C virus is one of the most common human pathogens. Modern therapy of hepatitis C is based almost solely on the use of interferon and its combination with the nucleoside analog ribavirin [Kozlov M. V., Polyakov K. M., Ivanov A. V., Biochemistry, 2006, v.71, No.9, pp. 1253-12594]. It should be noted that the therapy is not highly effective.

As for the development of therapeutic agents against hepatitis C virus, one of targets is NS3-serine proteinase having a zinc site that is important for the maintenance of its structural stability [Andrea Urbani, Renzo Bazzo, Maria Chiara Nardi, Daniel Oscar Cicero, Raffaele De Francesco, J. Biol. Chem., 1998, v.273, No. 30, pp. 18760-18769]. Some literary sources report that the inhibition or alteration of its activity by using compounds with zinc removal capacity is a promising strategy of controlling a hepatitis C virus disease.

The article of Timothy L. Tellinghuisen, Matthew S. Paulson, Charles M. Rice, J. Virology, 2006, v. 80, No. 15, pp. 7450-7458, teaches that metal chelators (EDTA and 1,10-phenanthroline), which were assessed relative to NS2/3 auto-cleavage, are effective inhibitors of proteases. In addition, there is information that the effect of 1,10-phenanthroline was associated, in this case, exactly with chelation of zinc.

The article of Sperandio D., Gangloff A. R., Litvak J. Goldsmith R., Bioorg. Med. Chem. Lett., 2002, v.12, No. 21, pp. 3129-3133, discloses screening of bisbenzimidazole compounds, aimed to seek for inhibitors of the hepatitis C virus NS3/NS4A serine protease, and mentions that said screening has also resulted in the identification of a corresponding powerful Zn²⁺-dependent inhibitor.

Another attractive target in the development of novel approaches to the therapy of hepatitis C can be the hepatitis C virus RNA-dependent RNA polymerase (viral protein NS5B) comprising a zinc-binding site [Timothy L. Tellinghuisen, Matthew S. Paulson, Charles M. Rice, J. Virology, 2006, v. 80, No. 15, pp. 7450-7458].

Currently known inhibitors of the hepatitis C virus RNA-dependent RNA polymerase can be conventionally divided into two main classes: nucleoside derivatives and various non-nucleoside inhibitors [Maria Bretner, Acta biochemica polonica, 2005, v.52, No. 1, pp. 57-70]. In addition, it has been found that pyrogallol derivatives inhibit the activity of the indicated enzyme. It is remarkable that the inhibition mechanism for pyrogallol derivatives is assumed to be based on the chelation of magnesium cations which are involved in the catalytic action at the phosphoryl transfer step [Kozlov M. V., Polyakov K. M., Ivanov A. V., Biochemistry, 2006, v.71, No. 9, pp. 1253-12594].

Diseases caused by herpes viruses are widely spread. Thus, there are known several human herpes viruses, such as herpes simplex viruses 1 and 2 (HSV-1 and HSV-2), cytomegalovirus (CV), varicella zoster virus, and Epstein-Barr virus. Their destructive actions on the central nervous system cause diseases, such as encephalitis and meningitis. There is a large interest in the studies of an effect of zinc-chelating compounds, for example diethylenetriaminopentaacetic acid, on the inhibition of human cytomegalovirus replication in vivo [Kanekiyo M., Itoh N., Mano M., Antiviral Res., 2000, v. 47, pp. 207-214].

However, it is also remarkable that herpes viruses, similar to the above-mentioned viruses, comprise proteins with a “zinc finger”-like moiety. Chemical modifications of the “zinc finger” structure can lead to the release of zinc and functional changes in the viral proteins [Yan Chen, Christine M. Livingston, Stacy D. Carrington-Lawrence, J. of Virology, 2007, v.81, No. 16, pp. 8742-8751].

The “zinc finger” structure can serve as a target for new generation antiviral therapy. A number of such compounds are already found. However, today their efficiency can be judged only on the basis of in vitro studies.

The aforesaid information suggests the importance of metal ions both for the normal functioning of cells and the organism as a whole and for the development of pathologies. The capacity of some compounds to chelate metal ions defines research strategy to develop medications effective in the treatment of various diseases, including viral diseases.

The article of Megan Whitnall, Jonathan Howard, Prem Ponka, “A class of iron chelators with a wide spectrum of potent antitumor activity that overcomes resistance to chemotherapeutics”, PNAS, 2006, v. 103, No. 40, pp. 14901-14906, discloses effective iron chelators which exhibit a high proliferative and anticancer activity similar to the activity of known cytostatics and which are potential for clinical trials.

Kik K., Szmigiero L., “Dexrazoxane (ICRF-187)—a cardioprotectant and modulator of some anticancer drugs”, Postepy Hig Med Dosw Online, 2006, v. 60, pp. 584-590, reports that some iron chelators can be used as “helpers” in anticancer therapy, owing to their cardioprotective action.

The chelation of copper ions inhibits angiogenesis and slows down the tumor growth [Yu Yu, Jacky Wong, David B. Loveioy, “Chelatorsat the cancer coalface: Desferrioxamine to Triapine and Beyond”, Clin. Cancer Res., 2006, v. 12, p. 6876-6883].

The zinc chelator, clioquinol, causes apoptosis of human cancer cells through the binding of Zn²⁺ [Haijun Yu, Yunfeng Zhou, Stuart E. Lind, “Clioquinol targets zinc to lysosomes in human cancer cells”, Biochem. J., 2009, v. 417, pp. 133-139].

Chelators are used as inhibitors of aldehyde dehydrogenase in the treatment of alcoholism [Shian S. G., Kao Y. R., Wu F. Y., Wu C. W., “Inhibition of invasion and angiogenesis by zinc-chelating agent disulfiram”, Mol. Pharmacol., 2003, v. 64(5), pp. 1076-84] and alcoholic cirrhosis, iron-excessive anemia, and porphyria cutanea tarda [Schroterova L., Kaiserova H., Baliharova V., “The effect of new lipophilic chelators on the activities of cytosolic reductases and P450 cytochromes involved in the metabolism of anthracycline as antibiotics: studies in vitro”, Physiol. Res., 2004, v. 53(6), pp. 683-691].

The activity mechanism of some antioxidants is chelating transition metal ions (Fe, Cu), which causes reduction in metal-dependent lipid peroxidation [Babizhayev M. A., Seguin Marie-C., Gueynej J., Evstigneev R. P., Ageyeva E. A., Zheltuchina G. A., “L-Carnosine(-alanyl-L-histidine) and carcinine f-alanylhistamine) act as natural antioxidants with hydroxyl-radical-scavenging and lipid-peroxidase activities”, Biochem. J., 1994, v. 304, pp. 509-516].

The use of antioxidants can lead to cataract resolution, elimination of retinal diseases, reduction in the need for insulin in diabetics, removal of skin pigmentation, and elimination of stroke sequelae symptoms. Chelation is also useful in the treatment of inflammatory diseases, such as osteoarthritis and rheumatoid arthritis [Zelenin K. N., “Chelators in medicine”, Soros Educational Journal, 2001, v.7, No.1, pp. 45-50].

Chelators can be used in medicine as chelating agents for transportation and easy extraction of arsenic, mercury, antimony, cobalt, zinc, chromium, and nickel from the organism [Zholnin A. V., “Complex compounds”, Chelyabinsk: ChGMA, 2000, p. 28].

Botulinus toxin is known to be inhibited by chelating zinc ions [Anne C., Blommaert A., “Thio-derivede disulfides as potent inhibitors of botulinum neurotoxin B: implications of zinc interaction”, Bioorg. Med. Chem., 2003, v. 11(21), pp. 4655-60]. In addition, chelation provides protection in gaseous gangrene therapy [Zelenin K. N., “Chelators in medicine”, Soros Educational Journal, 2001, v.7, No.1, pp. 45-50].

Chelation therapy is used in the treatment of neurodegenerative diseases, in particular Alzheimer's disease, to improve memory [Bossy-Wetzel E., Schwarzenbacher R., Lipton S. A., “Molecular pathways to neurodegeneration”, Nat. Med, 2004, v. 10, pp. 2-9]; Parkinson's disease [Kevin J. Barnham, Colin L. Masters, Ashley I. Bush, “Neurodegenerative diseases and oxidative stress”, Nature Reviews Drug Discovery, 2004, v. 3, pp. 205-214]; Wilson's disease [Yu Yu, Jacky Wong, David B. Lovejoy, “Chelators at the Cancer Coalface: Desferrioxamine to Triapine and Beyond”, Clin. Cancer Res., 2006, v. 12, pp. 6876-6883]; Huntington's disease [Whitnall M., Richardson D. R., “Iron: a new target for pharmacological intervention in neurodegenerative diseases”, Semin Pediatr Neurol, 2006, v. 13, pp. 186-197]; amyotrophic lateral sclerosis [Kevin J. Bernham, Colin L. Masters, Ashley I. Bush, “Neurodegenerative diseases and oxidative stress”, Nature Reviews Drug Discovery, 2004, v. 4], and prion diseases [Daniel L. Cox, Jianping Pan, Rajiv R. P. Singh, “A Mechanism for Copper Inhibition of Infection Prion Conversion”, Biophysical Journal, 2006, v. 91, L11-L13]. Chelators prevent the development of cancer [Megan Whitnall, “A class of iron chelators with a wide spectrum of potent antitumor activity that overcomes resistance to chemotherapeutics”, PNAS, 2006, v. 103, No. 40, p. 14901-14906].

Among known chelators, derivatives of heterocyclic compounds (for example, imidazole) comprising imido and amido groups are of special importance.

The article of M. A. Podyminogin, V. V. Vlassov, “Synthesis RNA-cleaving molecules mimicking ribonuclease A active center. Design and cleavage of tRNA transcrints”, Nucleic Acids Research, 1993, v. 21, No. 25, pp. 5950-5956, teaches a bis-histamine derivative of glutaric acid that can serve as a model of the active center of nucleases and exhibits a weak activity in the digestion of RNA molecules.

Elfriede Schuhmann et al. (see “Bis[platinum(II)] and Bis[Palladium (II)] complexes of α, \acute{ω}-Dicarboxylic Acid Bis(1,2,4-triaminobutane-N⁴)-Amides”, Inorg. Chem., 1995, v. 34, pp. 2316-2322) describe glutaric and adipic acid bis-histamine derivatives which are intermediates for the synthesis of complexes with platinum and palladium:

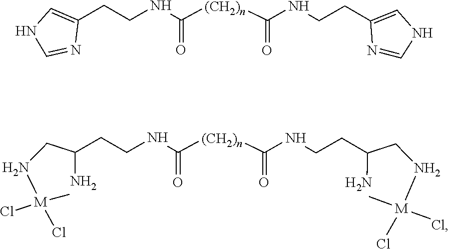

wherein n is 3-6 and 8; and

M is Pt and Pd.

The method of synthesis of N¹,N¹-glutaryl-bis(histamine), comprising contacting histamine dihydrocloride with glutaric acid dichloroanhydride in dimethylformamide in the presence of 4-fold excess of triethylamine is described in the article of Elfriede Schuhmann et al., “Bis[platinum(II)] and Bis[Palladium (II)] complexes of α, \acute{ω}-Dicarboxylic Acid Bis(1,2,4-triaminobutane-N⁴)-Amides”, Inorg. Chem., 1995, v. 34, pp. 2316-2322.

The inventors have found that a glutaric acid bis-histamine derivative, in particular N¹,N¹-glutaryl-bis(histamine), is capable of forming complexes with metal ions.

Thus, the objective of the present invention is to provide biocompatible heterocyclic metal ion chelators and the use thereof as a medicament for the treatment and/or prevention of various diseases by exploiting the capacity of the claimed compounds to chelate metal ions.

Another objective of the present invention is to provide simple methods for preparing such compounds by using available reactants.

### SUMMARY OF THE INVENTION

The present invention relates to dicarboxylic acid bisamide derivatives of general formula I:

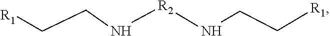

Wherein

R₁ is a 5-membered unsaturated heterocyclic group comprising from 1 to 2 heteroatoms selected from N and/or S, optionally condensed with a 6-membered unsaturated cyclic group;

R₂ is —C(O)—R₃—C(O)—, wherein R₃ is —(CH₂)ₙ— optionally substituted with one to two C₁-C₆alkyl groups or phenyl,

n is an integer from 0 to 4;

or a pharmaceutically acceptable salt thereof.

The present invention also relates to dicarboxylic acid bisamide derivatives of general formula I, which are able to chelate metal ions (Zn, Cu, Fe, Mg, Ca, etc.); and their use as an agent for the prevention and/or treatment of cardiac, viral, cancer, neurodegenerative and inflammatory diseases, diabetes, age-related diseases, diseases caused by microbial toxins, alcoholism and alcoholic cirrhosis, anaemia, porphyria cutanea tarda, and transition metal salt poisoning.

The present invention also relates to methods for preparing a compound of general formula I, comprising steps of:

contacting a dicarboxylic acid with a corresponding amine under heating; or

contacting a dicarboxylic acid with N-hydroxysuccinimide in the presence of N,N′-dicyclohexylcarbodiimide to form a corresponding bis-N-oxysuccinimide ester that is condensed with an amine; or

contacting a dicarboxylic acid diester with hydrazine hydrate, treating of the formed dihydrazide with sodium nitrite to form a corresponding azide that is condensed with an amine; or

heating a solution of the imide formed from the dicarboxylic acid with a corresponding amine in an organic solvent; or

contacting of a corresponding amine with a dicarboxylic acid in a molar ratio of 2:1 in the presence of a condensing agent.

The claimed methods for preparing dicarboxylic acid heterocyclic bis-derivatives of general formula I are simple in implementation, conducted under quite mild conditions, are free of by-products, readily reproducible, and provide target products with a high yield (up to 82%) and of a high purity.

### DETAILED DESCRIPTION OF THE INVENTION

Preferred compounds according to the invention are compounds of general formula I:

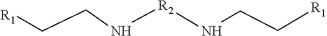

wherein R₁ is a group selected from:

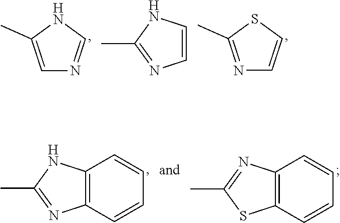

R₂ is a group selected from: —C(O)—(CH₂)₀—(CO)—, (CH₂)₁—(CO)—, —C(O)—(CH₂)₂—(CO)—, —C(O)—(CH₂)₃—(CO)—, —C(O)—(CH₂)₄—(CO)—C(O)—CH₂—CH(CH₃)—CH₂—C(O)—, —C(O)—CH₂—C(CH₃)₂—CH₂—C(O)—, or a group selected from:

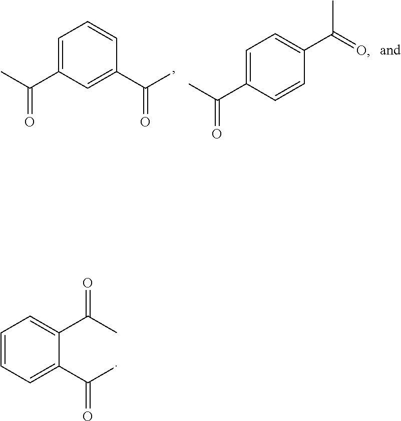

The most preferred compounds of the present invention are compounds given in Table 1.

TABLE 1

| Number of | |
|---|---|
| a compound | Structure |
| 1 | 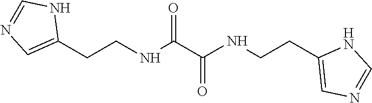 [SMILES: O=C(NCCc1cnc[nH]1)C(=O)NCCc1cnc[nH]1] |
| 2 | 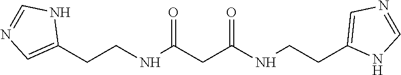 [SMILES: O=C(CC(=O)NCCc1cnc[nH]1)NCCc1cnc[nH]1] |
| 3 | 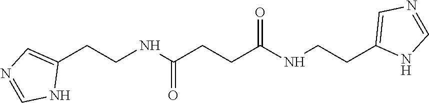 [SMILES: O=C(CCC(=O)NCCc1cnc[nH]1)NCCc1cnc[nH]1] |
| 4 | 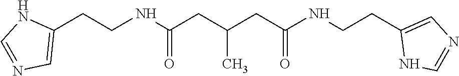 [SMILES: CC(CC(=O)NCCc1cnc[nH]1)CC(=O)NCCc1cnc[nH]1] |
| 5 | 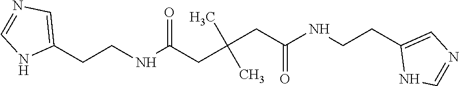 [SMILES: CC(C)(CC(=O)NCCc1cnc[nH]1)CC(=O)NCCc1cnc[nH]1] |
| 6 | 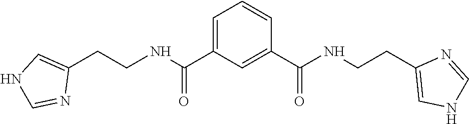 [SMILES: O=C(NCCc1c[nH]cn1)c1cccc(C(=O)NCCc2c[nH]cn2)c1] |
| 7 | 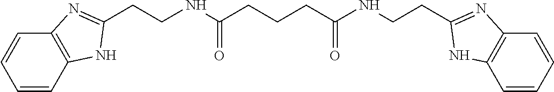 [SMILES: O=C(CCCC(=O)NCCc1nc2ccccc2[nH]1)NCCc1nc2ccccc2[nH]1] |
| 8 | 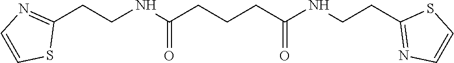 [SMILES: O=C(CCCC(=O)NCCc1nccs1)NCCc1nccs1] |
| 9 | 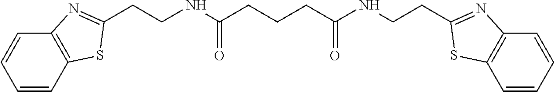 [SMILES: O=C(CCCC(=O)NCCc1nc2ccccc2s1)NCCc1nc2ccccc2s1] |
| 10 | 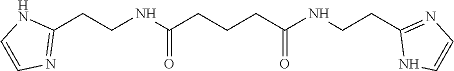 [SMILES: O=C(CCCC(=O)NCCc1ncc[nH]1)NCCc1ncc[nH]1] |

Pharmaceutically acceptable salts of the compounds according to the present invention may be selected from additive salts of organic acids (for example, formiate, acetate, maleate, tartrate, methanesulfonate, benzenesulfonate, toluenesulfonate, etc.), additive salts of inorganic acids (for example, hydrochloride, hydrobromide, sulphate, phosphate, etc.), and salts with amino acids (for example, an asparaginic acid salt, a glutamic acid salt, etc.), preferably chlorohydrates and acetates.

The most preferred known compounds that can be used in the pharmaceutical composition and method for the treatment according to the present invention are glutarimide derivatives represented in Table 2.

TABLE 2

| Number of | |
|---|---|
| a compound | Structure |
| 11 | 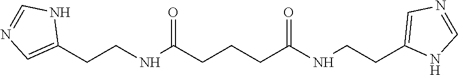 [SMILES: O=C(CCCC(=O)NCCc1cnc[nH]1)NCCc1cnc[nH]1] |
| 12 | 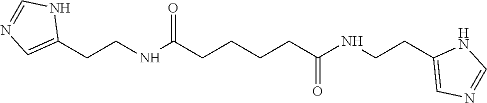 [SMILES: O=C(CCCCC(=O)NCCc1cnc[nH]1)NCCc1cnc[nH]1] |

The compounds according to the present invention can be prepared by a method comprising condensing a dicarboxilic acid of general formula II:

R₄O—C(O)—R₃—C(O)—OR₄,

wherein R₃ is —(CH₂)ₙ— optionally substituted with one to two C₁-C₆ alkyl groups or phenyl;

n is an integer from 0 to 4, and

R₄ is hydrogen or C₁-C₆alkyl;

with an amine of general formula III:

NH₂—(CH₂)₂—R₁,

wherein R₁ is a 5-membered unsaturated heterocyclic group comprising from 1 to 2 heteroatoms selected from N and/or S, optionally condensed with a 6-membered unsaturated cyclic group;

under heating, optionally in the presence of a solvent.

It is preferable to use of dimethyl ether and to heat to 150-170° C.; and it is more preferable to perform the condensation under boiling.

The solvent can be diglyme or an alcohol, more preferable isoamyl alcohol.

Another method for producing dicarboxylic acid bisamide derivatives of general formula I is a method comprising contacting a dicarboxylic acid of general formula II:

R₄O—C(O)—R₃—C(O)—OR₄,

wherein R₃ is —(CH₂)ₙ— optionally substituted with one to two C₁-C₆alkyl groups or phenyl,

n is an integer from 0 to 4, and

R₄ is hydrogen,

with N-hydroxysuccinimide in the presence of N,N′-dicyclohexylcarbodiimide to form a corresponding bis-N-oxysuccinimide ester of general formula IV:

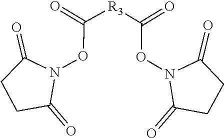

that is condensed with an amine of general formula III:

NH₂—(CH₂)₂—R₁,

wherein R₁ is a 5-membered unsaturated heterocyclic group comprising from 1 to 2 heteroatoms selected from N and/or S, optionally condensed with a 6-membered unsaturated cyclic group.

It is preferable to provide cooling to a temperature of 0-5° C.

Another method for producing dicarboxylic acid bisamide derivatives of general formula I is a method comprising contacting a dicarboxylic acid ester of general formula II:

R₄O—C(O)—R₃—C(O)—OR₄,

wherein R₃ is —(CH₂)ₙ— optionally substituted with one to two C₁-C₆alkyl groups or phenyl,

n is an integer from 0 to 4, and

R₄ is C₁-C₆alkyl,

with hydrazine hydrate in an organic solvent to form bishydrazide of general formula V:

H₂N—NH—C(O)—R₃—C(O)—NH—NH₂,

treating the bishydrazide with sodium nitrite in an acidic medium at a temperature of about 0° C. to form bisazide of general formula VI:

N⁻—N⁺═N—C(O)—R₃—C(O)—N═N⁺—N⁻,

condensing the bisazide with an amine of general formula III:

NH₂—(CH₂)₂—R₁

wherein R₁ is a 5-membered unsaturated heterocyclic group comprising from 1 to 2 heteroatoms selected from N and/or S, optionally condensed with a 6-membered unsaturated cyclic group, in an organic solvent.

An alcohol is preferable to be used as an organic solvent, and isopropanol is more preferable. The method is simple but is used when the number of methylene units in the initial dicarboxylic acid is more or equal to three since bisazides thereby obtained for corresponding acids with a lower number of methylene groups are not stable.

Dicarboxylic acid bisamide derivatives of general formula I also can be prepared by a method comprising condensing an imide of general method VII:

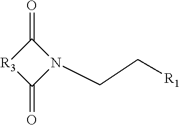

wherein R₃ is —(CH₂)ₙ— optionally substituted with one or two C₁-C₆alkyl groups, and

n is an integer from 0 to 4,

with an equimolar amount of an amine of general formula III:

NH₂—(CH₂)₂—R₁,

wherein R₁ is a 5-membered unsaturated heterocyclic group comprising from 1 to 2 heteroatoms selected from N and/or S, optionally condensed with a 6-membered unsaturated cyclic group,

in an organic solvent under heating.

An alcohol is preferable to be used as an organic solvent, and isopropanol is more preferable. The condensation is preferable to be performed under boiling.

Another method of the present invention is a method for producing dicarboxylic acid bisamide derivatives of general formula I, comprising contacting a dicarboxylic acid of general formula II:

R₄O—C(O)—R₃—C(O)—OR₄,

wherein R₃ is —(CH₂)ₙ— optionally substituted with one to two C₁-C₆alkyl groups or phenyl,

n is an integer from 0 to 4, and

R₄ is hydrogen,

with an amine of general formula III:

NH₂—(CH₂)₂—R₁,

wherein R₁ is a 5-membered unsaturated heterocyclic group comprising from 1 to 2 heteroatoms selected from N and/or S and optionally condensed with a 6-membered unsaturated cyclic group;

in a molar ratio of 1:2-2.5 in a tetrahydrofuran solution in the presence of a condensing agent, preferably carbonyldiimidazole.

The present invention also relates to a medicament and a pharmaceutical composition comprising a dicarboxylic acid bisamide derivative of general formula I, and to a method comprising administering a dicarboxylic acid bisamide derivative of general formula I to treat and/or prevent in a human and an animal viral diseases, such as diseases caused by hepatitis C virus, human papilloma virus, HIV or oncogenic RNA viruses, such as leukemia virus; cardiovascular diseases, including a disease caused by cytostatic cardiotoxicity, cholesterol accumulation, and hypertension; diseases associated with metal-dependent free-radical oxidation reactions, including age-related diseases, such as cataract, retinal disease, and skin pigmentation; stroke sequelae; atherosclerosis; inflammatory diseases, such as osteoarthritis, rheumatoid arthritis;

diabetes and its cardiovascular complications; neurodegenerative diseases, including Alzheimer's disease, Parkinson's disease, Wilson's disease, Huntington's disease, amyotrophic lateral sclerosis, and prion diseases;

oncologic diseases; diseases caused by microbial toxins, in particular botulism or gaseous gangrene;

alcoholism and alcoholic cirrhosis; iron-excessive anaemia, porphyria cutanea tarda; and transition metal salt poisoning.

The compounds according to the present invention are administered in an effective amount that provides a desired therapeutic effect.

The compounds of general formula I can be administered orally, topically, parenterally, intranasally, by inhalation, and rectally in a unit dosage form comprising non-toxic pharmaceutically acceptable carriers. The term “oral administration” used in the present invention means subcutaneous, intravenous, intramuscular or intrathoric injection or infusion.

The compounds according to the present invention can be administered to a patient at a dose of from 0.1 to 100 mg/kg of body weight once daily, preferably at a dose of from 0.25 to 25 mg/kg one or more times a day.

In addition it should be noted that a particular dose for a particular patient depends on many factors, including the activity of a certain compound, patient's age, body weight, gender, general health condition and diet, the time and route of administration of a pharmaceutical agent and the rate of its excretion from the body, a specific combination of drugs, and the severity of a disease in an individual to be treated.

The pharmaceutical compositions according to the present invention comprise a compound of general formula (I) in an amount effective to achieve a desired technical result, and can be administered in a unite dosage form (for example, in a solid, semi-solid, or liquid form) comprising the compounds according to the present invention as an active agent in a mixture with a carrier or an excipient suitable for intramuscular, intravenous, peroral and sublingual administration, administration by inhalation, intranasal and intrarectal administration. The active ingredient can be in a composition together with conventional nontoxic pharmaceutically acceptable carriers suitable for the manufacture of solutions, tablets, pills, capsules, coated pills, emulsions, suspensions, ointments, gels, and any other dosage forms.

As an excipient, various compounds can be used, such as saccharides, for example, glucose, lactose, of sucrose;

mannitol or sorbitol; cellulose derivatives; and/or calcium phosphates, for example, tricalcium phosphate or calcium hydrophosphate. As a binder, the following compounds can be used, such as a starch paste (for example, corn, wheat, rice, or potato starch), gelatin, tragacanth, methylcellulose, hydroxypropylmethylcellulose, sodium carboxymethylcellulose, and/or polyvinylpyrrolidone. Optionally used disintegrants are the above-mentioned starches and carboxymethylstarch, crosslinked polyvinylpyrrolidone, agar-agar, or alginic acid or a salt thereof, such as sodium alginate.

Additives that can be optionally used are flowability-control agents and lubricants, such as silicon dioxide, talc, stearic acid and salts thereof, such as magnesium stearate or calcium stearate, and/or propylene glycol.

The core of a coted pill is usually coated with a layer that is resistant to the action of gastric acid. For this purpose a concentrated solution of saccharides can be used, wherein said solutions can optionally comprise gum arabic, talc, polyvinylpyrrolidone, polyethylene glycol, and/or titanium dioxide, and suitable organic solvents or a mixture thereof.

Stabilizers, thickening agents, colorants, and fragrances also can be used as additives.

As an ointment base, there are usable hydrocarbon ointment bases, such as white Vaseline and yellow Vaseline (Vaselinum album and Vaselinum flavum, respectively), Vaseline oil (Oleum Vaselini), and white ointment and liquid ointment (Unguentum album and Unguentum flavum, respectively), wherein solid paraffin or wax can be used as an additive providing a firmer texture; absorptive ointment bases, such as hydrophilic Vaseline (Vaselinum hydrophylicum), lanoline (Lanolinum), and cold cream (Unguentum leniens); water-removable ointment bases, such as hydrophilic ointment (Unguentum hydrophylum); water-soluble ointment bases, such as polyethylene glycol ointment (Unguentum Glycolis Polyaethyleni); bentonite bases; and others.

A base for gels may be selected from methylcellulose, sodium caboxymethylcellulose, oxypropylcellulose, polyethylene glycol or polyethylene oxide, and carbopol.

A base for suppositories can be a water-insoluble base such as cocoa butter; a water-soluble or water-miscible base, such as gelatin-glycerol or polyethylene oxide; or a combined base, such as a soap-glycerol base.

In preparing a unit dosage form, the amount of an active agent used in combination with a carrier can vary depending on a recipient to be treated and on a particular route of administration of a therapeutic agent.

For example, when the compounds according to the present invention are used in the form of a solution for injection, the amount of the active agent in this solution is up to 5 wt. %. A diluent may be selected from a 0.9% sodium chloride solution, distilled water, a Novocain solution for injection, Ringer's solution, a glucose solution, and specific solubilizing adjuvants. When the compounds according to the present invention are administered in tablet or suppository form, their amount is up to 200 mg per unit dosage form.

Dosage forms according to the present invention are prepared by conventional procedures, such as blending, granulation, forming coating pills, dissolution, and lyophilization.

The detailed description of the compounds according to the present invention, their preparation, and studies of their activity are disclosed in the following examples that are intended for purposes of illustration only and are not intended to limit the scope of the invention.

### Examples of Synthesis of Glutarimide Derivatives of General Formula I

### Means and Methods

Identity of obtained compounds were assessed by the thin-layer chromatography (TLC) method on plates “Kieselgel 60 F254” (“Merck”, German) in a solvent system of pyridine-acetic acid-water (20:6:11) (System A); A:ethylacetate (3:1) (1), and chloroform-methanol (9:1) (2).

Paper electrophoresis (electrophoresis paper of Kondopozhskii TsBK, 120×320 mm) was performed in a buffer (pH 5.1) consisting of pyridine-acetic acid-water (12:10:1000) in a chamber (150×320×150 mm) with a gradient of 15 V/cm for 1.5 hours.

Chromatograms and electrophoregrams were treated with chloro-tetramethylbenzene reagent and Pauly's reagent.

The melting point was measured with a melting point apparatus (Plant of lab. equipment, Klin, Russia).

Fourier IR spectra were recorded on a “Magna 750” spectrometer with KBr tablets (“Nicolet” (US)).

¹H NMR spectra were registered on Bruker AMX-400 (German) and DPX-400 (German) spectrometers.

High-resolution mass-spectra were obtained on a time-of-flight-assisted mass-spectrometer by the method of matrix laser-desorption ionization with 2,5-dihydroxybensoic acid used as a matrix, on an Ultraflex mass spectrometer (“Bruker”, German).

Shimadzu Analytical HPLC SCL10Avp LC/MS system was used for the analysis of multicomponent mixtures on a mass spectrometer PE SCIEX API 165 (150) (Canada).

Analytical-scale reversed phase HPLC was performed on a Shimadzu HPLC chromatograph under the following conditions: Luna C18 (2) 100 A column, 250×4.6 mm (No. 599779-23), an elution gradient in a system of a phosphate buffer solution (pH 3.0):methanol (condition A); and on “System Gold” chromatographer (Beckman, US) with UV-detector (λ=220 nm), Phenomenex “Gemini” C-18 column, 150×2.0 mm, a mobile phase flow gradient in a 0.05 M phosphate buffer (pH 3.0), 90% MeCN in water, an elution rate of 0.25 ml/min, and 10 μl injection (condition B).

### EXAMPLE 1

Bis-1,5-(N^{β}-histaminyl) glutaric acid; (N,N′-bis-[2-(1H-imidazol-4-yl)ethyl]pentanediamide (Compound 11).

Histamine (8 g; 0.072 mol) was added to glutaric acid dimethyl ester (5 g; 0.031 mol) and heated at 170° C. for 3.5-4 hours until the evaporation of methyl alcohol was completed. The completeness of the reaction was checked by a TLC or electrophoresis method. Then the reaction mixture was suspended in isopropyl alcohol and allowed to stand for 24 hours at +4° C. The product was separated, washed with isopropyl alcohol, and dried. Yield was 6.8 g (69%). R_{f} 0.42 (1). E⁺49 mm. M.p. 166-168° C. LC/MS: an individual peak at a retention time of 0.3 min, [M+H]⁺=319. HPLC under condition A: an individual peak at a retention time of 3.58 min. ¹H NMR (400.13 MHz, DMSO-d₆, δ, m.d., J/Hz): 1.70 (quint, 2H, CH₂CH₂CH₂, J=7.3 Hz), 2.03 (t, 4H, CH₂CH₂CH₂, J=7.3 Hz), 2.61 (t, 4H, CCH₂CH₂N, J=7.5 Hz), 3.26 (quint, 4H, CCH₂CH₂N, J=7.5 Hz), 6.76 (b.s, 2H, CCH), 7.50 (s, 2H, NCHN), 7.94 (b.t., 2H, NH). Fourier-IR spectrum (in a KBr-table, ν, cm⁻¹): 1651 (ν C═O amide I), 1580 (δ NH amide II), 1425 (—CH₂—CO—).

Found, %: C, 56.40; H, 6.86; N, 26.31. C₁₅H₂₂N₆O₂. Calculated, %: C, 56.59; H, 6.96; N, 26.40.

### EXAMPLE 2

### Bis-1,4-(N^{β}-histaminyl)succinic acid; (N,N′-bis-[2-(1H-imidazol-4-yl)ethyl]butanediamide) (Compound 3)

Histamine (12.2 g; 0.11 mol) was added to succinic acid (5.5 g; 0.046 mol) and heated at 170° C. for 3.5-4 hours until the evaporation of methyl alcohol was completed. The completeness of the reaction was checked by a TLC or electrophoresis method. Then the reaction mass was suspended in 50 ml of water and allowed to stand for 24 fours at +4° C. The product was separated, washed with water, and dried. Yield was 10.7 g (76%). R_{f} 0.51 (1). E⁺51 mm. M.p. 230-231° C. [M+H]⁺304.95. ¹H-NMR (D₂O): δ, m.d.: 2.48 (s, 4H, CH₂-Suc), 2.80-2.84 (t, 4H, β-CH₂-HA), 3.44-3.48 (t, 4H, α-CH₂-HA), 6.97 (s, 2H, 5-CH-Im), 7.78 (s, 2H, 2-CH-Im). Fourier-IR spectrum (in a KBr-table, ν, cm⁻¹): 1634 (ν C═O amide I), 1583 (δ NH amide II), 1429 (—CH₂—CO—). Found, %: C, 55.40; H, 6.66; N, 27.31. C₁₄H₂₀N₆O₂. Calculated, %: C, 55.25; H, 6.62; N, 27.61.

### EXAMPLE 3

### Bis-1,3-(N^{β}-histaminyl)malonic acid; (N,N′-bis-[2-(1H-imidazol-4-yl)ethyl]propanediamide) (Compound 2)

Histamine (9.0 g; 0.081 mol) was added to a malonic acid diethyl ester (6.4 g; 0.039 mol) and heated at 170° C. for 2.5-3 hours until the evaporation of methyl alcohol was completed. The completeness of the reaction was checked by a TLC or electrophoresis method. Then the reaction mass was suspended in 20 ml of water and allowed to stand for 72 fours at +4° C. The product was separated, washed with isopropyl alcohol, and dried. Yield was 5.0 g (44%). R_{f} 0.41 (1). E⁺57 mm. M.p. 187-188° C. ¹H-NMR (D₂O): δ, m.d.: 2.74-2.78 (t, 4H, β-CH₂-HA), 3.15 (s, 2H, CH₂-Mal), 3.41-3.44 (t, 4H, α-CH₂-HA), 6.78 (s, 2H, 5-CH-Im), 7.66 (s, 2H, 2-CH-Im). Fourier-IR spectrum (in a KBr-table, ν, cm⁻¹): 1643 (ν C═O amide I), 1574 (δ NH amide II), 1426 (—CH₂—CO—). Found, %: C, 53.24; H, 6.51; N, 28.56. C₁₃H₁₈N₆O₂. Calculated, %: C, 53.78; H, 6.25; N, 28.95.

### EXAMPLE 4

### Bis-1,2-(N^{β}-histaminyl) oxalic acid; (N,N′-bis-[2-(1H-imidazol-4-yl)ethyl]ethanediamide) (Compound 1)

Histamine (12.2 g; 0.11 mol) was added to a solution of an oxalic acid diethyl ester (7.3 g; 0.05 mol) in isoamyl alcohol (35 ml) and boiled for 4 hours. The completeness of the reaction was checked by a TLC or electrophoresis method. The reaction mixture was allowed to stand for 16 hours at +4C°. The residue was separated, washed with isopropyl alcohol, and dried. Yield was 10 g (72%). R_{f} 0.55 (1). E⁺55 mm. M.p. 235-236° C. ¹H-NMR (DMSO): δ, m.d.: 2.66-2.72 (t, 4H, (β-CH₂-HA), 3.33-3.41 (m, 4H, α-CH₂-HA), 6.81 (s, 2H, 5-CH-Im), 7.54 (s, 2H, 2-CH-Im), 8.78-8.84 (t, 2H, NH—CO). Fourier-IR spectrum (in a KBr-table, ν, cm⁻¹): 1651 (ν C═O amide I), 1566 (δ NH amide II), 1425 (—CH₂-CO—). Found, %: C, 52.24; H, 5.51; N, 29.97. C₁₂H₁₆N₆O₂. Calculated, %: C, 52.17; H, 5.84; N, 30.42.

### EXAMPLE 5

### Bis-1,3-(N^{β}-histaminyl) isophthalic acid (Compound 6)

Isophthalic acid (5 g; 30 mmol) and N-hydroxysuccinimide (7.93; 69 mmol) were dissolved in dimethylformamide (30 ml). A cooled solution of N,N′-dicyclohexylcarbodiimide (14.24 g; 69 mmol) in dimethylformamide (10 ml) was added to the resulting solution cooled to 5° C., and the reaction mixture was allowed to stand for a night at 5° C. Urea was separated, washed with 15 ml of dimethylformamide, and dried. Based on the mass of the resulting urea, the yield of the reaction was considered to be equal to 80%. The solution of an isophthalic acid N-hydroxysuccinimide diester was cooled to 5° C., and then melted histamine (6.84 g; 61.6 mmol) was fed thereto batchwise. The reaction mixture was allowed to stand for 2 hours. The solvent was removed under vacuum. The yellow oil residue was dissolved in 120 ml of water and passed through a column with Amberlite IRA-96 (27×115). The fractions comprising the product were combined. Precipitated crystals of the target product were separated. The obtained crude product was recrystallized from a mixture of isopropanol (35 ml) and water (10 ml). The crystals of the product were filtrated, washed with water (3×30 ml) and isopropanol (2×13 ml), and dried. The target product was obtained in the form of white crystals. Yield was 1.55 g (18% based on histamine, or 14% based on isophthalic acid). R_{f} 0.42 (1). M.p. 218-219° C. E⁺40 mm. MS: [M+1]⁺353. ¹H-NMR: (300 MHz, DMSO-d₆): δ, m.d.: 2.74-2.79 (t, 4H, β-CH₂-Ha), 3.46-3.52 (t, 4H, α-CH₂-Ha), 6.81 (s, 2H, 5-CH-Im), 7.52-7.56 (m, 3H, ArH⁺²—CH-Im), 7.92-7.95 (d, 2H, ArH), 8.28 (s, 1H, ArH) , 8.65 (b.s., 2H, —C(O)—NH—), 11.80 (b.s., 2H, —NH—). HPLC under condition B: an individual peak at a retention time of 12.5 min.

### EXAMPLE 6

### Bis-1,5-(N^{β}-histaminyl)glutaric acid; (N,N′-bis-[2-(1H-imidazol-4-yl)ethyl]pentanediamide) (Compound 11)

PCl₃ (8 ml) was added batchwise to a solution of glutaric acid (13.2 g; 0.10 mol) in methanol (50 ml) under cooling and stirring. The solvent was removed from the reaction mixture under vacuum. The resulting residue was distilled off under vacuum. The amount of the resulting glutaric acid dimethyl ester with a boiling point of 110-112° C. was 14.7 (92%).

Hydrazine hydrate (7.5 ml) was added to isopropyl alcohol (20 ml), heated to a boiling point and then the glutaric acid dimethyl ester (8 g; 0.05 mol) was added thereto dropwise, and the reaction mixture was allowed to stand for 16 hours at +20° C. The precipitated product was separated, washed with isopropyl alcohol, and dried. The amount of glutaric acid dihydrazide with a melting point of 210-212° C. was 7.3 (91%).

Sodium nitrate (1.45 g; 0.021 mol) was added batchwise under stirring to a solution of the glutaric acid dihydrazide (1.61 g; 0.010 mol) in a mixture of ice (20 g) with hydrochloric acid (2.5 ml) and previously cooled chloroform (10 ml). The chloroform layer was separated, washed with water (10 ml), cooled to +4° C., and then isopropanol (5 ml) was added to a cooled histamine solution (2.6 g; 0.023 mol). The reaction mixture was allowed to stand for 2 hours at +20° C., and then the solvent was removed under vacuum. The resulting residue was dissolved in water (20 ml) and passed through a column with 50 ml of ion-exchange resin KU-2-20 (H⁺-form). The resin was washed with a 0.5% ammonia solution, collecting histamine-free fractions of the product. Eluates were combined, the solvent was removed under vacuum, and the resulting residue was recrystallized from isopropanol (2 ml) and dried. The yield was 1.8 g (55%). R_{f} 0.42 (1). E⁺49 mm. M.p. 166-168° C. ¹H-NMR (D₂O): δ, m.d.: 1.73-1.78 (m, 2H, β-CH₂-Glt), 2.10-2.15 (t, 4H, α-CH₂-Glt), 2.78-2.82 (t, 4H, β-CH₂-HA), 3.43-3.48 (t, 4H, α-CH₂-HA), 6.94 (s, 2H, 5-CH-Im), 7.69 (s, 2H, 2-CH-Im). Fourier-IR spectrum (in a KBr table, ν, cm⁻¹): 1651 (ν C═O amide I), 1580 (δ NH amide II) , 1425 (—CH₂—CO—). Found, %: C, 56.40; H, 6.86; N, 26.31. C₁₅H₂₂N₆O₂. Calculated, %: C, 56.59; H, 6.96; N, 26.40.

### EXAMPLE 7

### Bis-1,5-(N^{β}-histaminyl)adipic acid; (N,N′-bis-[2-(1H-imidazol-4-yl)ethyl]hexanediamide) (Compound 12)

Phosphorus trichloride (4 ml) was added dropwise under cooling to a solution of adipic acid (7.40; 0.05 mol) in methanol (25 ml). The solvent was removed from the reaction mixture under vacuum, and the resulting residue was distilled off under vacuum. The amount of the resulting adipic acid dimethyl ester with a boiling point of 115-117° C. was 7.5 (86%).

The adipic acid dimethyl ester (7.5 g; 0.04 mol) was added dropwise to a solution of isopropyl alcohol (20 ml) and hydrazine hydrate (5 ml), heated to a boiling point, then the reaction mixture was allowed to stand for 16 hours at +20° C. The precipitate was separated, washed with isopropyl alcohol, and dried. The amount of the resulting adipic acid dihydrazide with a melting point of 182-182.5° C. was 6.2 (83%).

Sodium nitrate (1.8 g; 0.026 mol) was added portionwise under stirring to a solution of the adipic acid dihydrazide (2.2 g; 0.013 mol) in a mixture of ice (30 g) with hydrochloric acid (3 ml) and cooled chloroform (15 ml). The chloroform layer was separated, washed with water (10 ml), and cooled to +4° C., then isopropanol (8 ml) was added to the cooled histamine solution (3 g; 0.027 mol). The completeness of the reaction was checked by a TLC or electrophoresis method. The reaction mixture was allowed to stand for 24 hours at +4° C., and the residue was separated, washed with isopropyl alcohol, recrystallized from water, and dried. The yield was 2.8 g (65%). R_{f} 0.48 (1). E⁺45 mm. M.p. 184-186° C. [M+H]⁺332.92. ¹H-NMR (D₂O): δ, m.d.: 1.37-1.41 (m, 4H, β-CH₂-Adip), 2.11-2.15 (m, 4H, α-CH₂-Adip), 2.74-2.78 (t, 4H, β-CH₂-HA), 3.40-3.44 (t, 4H, α-CH₂-HA), 6.88 (s, 2H, 5-CH-Im), 7.63 (s, 2H, 2-CH-Im). Fourier-IR spectrum (in a KBr table, ν, cm⁻¹): 1646 (ν C═O amide I), 1581 (δ NH amide II), 1425 (—CH₂—CO—). Found, %: C, 57.45; H, 7.15; N, 24.97. C₁₆H₂₄N₆O₂. Calculated, %: C, 57.81; H, 7.28; N, 25.28.

The following compounds were prepared by the above-disclosed method:

| Number of | | |
|---|---|---|
| a compound | Structural formula | Physical and chemical data |
| Compound 8 | 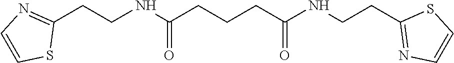 [SMILES: O=C(CCCC(=O)NCCc1nccs1)NCCc1nccs1] | MS: [M + 1]⁺ = 353. HPLC: under condition B, an individual peak at a retention time of 16.1 min. ¹H-NMR (400 |
| | | MHz, DMSO-d₆): δ, m.d.: |
| | | 1.63-1.75 (2H, m, —CH₂), |
| | | 1.96-2.11 (4H, t, J = 7.46, |
| | | 2CH₂), 3.06-3.16 (4H, t, |
| | | J = 6.97, CH₂), 3.35-3.46 |
| | | (4H, dd, J1 = 6.61, J2 = 6.11, |
| | | CH₂), 7.66-7.74 (2H, d, |
| | | J = 3.18, Ar), 7.66-7.74 |
| | | (2H, d, J = 3.18, Ar), 7.9- |
| | | 8.0 (2H, m, NH). |
| Compound 10 | 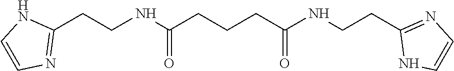 [SMILES: O=C(CCCC(=O)NCCc1ncc[nH]1)NCCc1ncc[nH]1] | LC/MS: an individual peak at a retention time of 0.3 min. [M + H]⁺ = 319. HPLC under condition A: an individual peak at a retention time |
| | | of 4.12 min. ¹H NMR (400.13 |
| | | MHz, DMSO-d₆, δ, m.d., |
| | | J/Hz): 1.69 (quint, 2H, |
| | | CH₂CH₂CH₂, J = 7.3 Hz), 2.01 |
| | | (t, 4H, CH₂CH₂CH₂, J = 7.3 |
| | | Hz), 2.73 (t, 4H, CCH₂CH₂N, |
| | | J = 7.5 Hz), 3.31 (quint, |
| | | 4H, CCH₂CH₂N, J = 7.5 Hz), |
| | | 6.87 (s, 4H, CHN), 8.00 |
| | | (b.t., 2H, NH). |

### EXAMPLE 8

### Bis-1,4-(N^{β}-histaminyl)succinic acid; (N,N′-bis-[2-(1H-imidazol-4-yl)ethyl]butanediamide) (Compound 3)

Succinimide histamine (0.15; 0.78 mmol) was added to a solution of histamine (0.10 g; 0.9 mmol) in isopropyl alcohol (3 ml) and heated under boiling for 2.5-3 hours. The completeness of the reaction was checked by a TLC or electrophoresis method. Then the reaction mixture was cooled to room temperature and allowed to stand for 26 fours at +4° C. The product was separated, washed with isopropyl alcohol, and dried. Yield was 0.20 g (81%). R_{f} 0.44 (1). E⁺51 mm. M.p. 231° C. [M]⁺304.12. ¹H-NMR (D₂O): δ, m.d.: 2.48 (s, 4H, CH₂-Suc), 2.80-2.84 (t, 4H, β-CH₂-HA), 3.44-3.48 (t, 4H, α-CH₂-HA), 6.97 (s, 2H, 5-CH-Im), 7.78 (s, 2H, 2-CH-Im). Fourier-IR spectrum (in a KBr table, ν, cm⁻¹): 1634 (ν C═O amide I), 1583 (δ NH amide II), 1429 (—CH₂—CO—). Found, %: C, 6.57; N, 27.18. C₁₄H₂₀N₆O₂. Calculated, %: C, 55.25; H, 6.62; N, 27.61.

### EXAMPLE 9

### Bis-1,5-(N-ethylamino-2-benzimidazolyl)glutaric acid; (N,N′-bis-[2-(1--benzimidazol-2-yl) ethyl]pentanediamide) (Compound 7)

Carbonyldiimidazole (23.4 g; 0.145 mol) was added to a solution of glutaric acid (8 g; 0.06 mol) in anhydrous tetrahydrofuran (500 ml) and stirred for 2 hours. Then benzimidazolyl ethylamine (21.6 g; 0.133 mol) was added to the reaction mixture. The resulting solution was stirred for 3 hours and allowed to stand at room temperature. The residue of the product was separated, washed with water, and dried. The yield was 21 g (82.9%). R_{f} 0.21 (2). M.p. 170-170.5° C. [M]⁺419. ¹H-NMR (DMSO): δ, m.d.: 1.61-1.76 (m, 2H, CH₂), 1.95-2.07 (t, 2H, CH₂), 2.9-3.0 (t, 4H, 2CH₂), 3.45-3.55 (quint, 4H, 2CH₂), 7.06-7.14 (m, 4H, Ar), 7.39-7.54 (m, 4H, Ar), 7.98-8.08 (t, 2H, Ar), 11.6-12.7 (s, 2H, NH). HPLC under condition B: an individual peak at a retention time of 26.6 min.

The following compounds were prepared by the above-disclosed method:

| Number of | | |
|---|---|---|
| a compound | Structural formula | Physical and chemical data |
| Compound 9 | 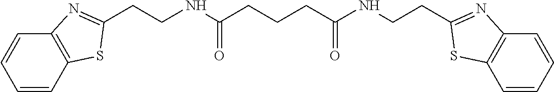 [SMILES: O=C(CCCC(=O)NCCc1nc2ccccc2s1)NCCc1nc2ccccc2s1] | MS: [M + 1]⁺ = 453. ¹H-NMR (400 MHz, DMSO-d₆): δ, m.d.: 1.65-1.77 (2H, m, CH₂), 2.0-2.1 (2H, t, J = 7.46, CH₂), 3.16-3.26 (4H, t, J = 6.97, 2CH₂), 3.45-3.56 |
| | | (4H, quint, J1 = 6.48, |
| | | J2 = 5.87, 2CH₂), 7.35-7.44 |
| | | (1H, m, Ar), 7.44-7.52 |
| | | (1H, m, Ar), 7.9-7.96 (2H, |
| | | d, Ar, J = 7.82), 7.96-8.02 |
| | | (2H, t, NH, J = 5.5). 8.02- |
| | | 8.07 (2H, d, Ar, J = 7.82). |
| | | HPLC under condition B: an |
| | | individual peak at a |
| | | retention time of 18.7 min |

### Study of the Chelating Ability of the Claimed Compounds

The following studies have revealed that the claimed compounds of general formula I are able to form complexes with or chelate metal ions. They comprise several functional groups that can serve as donors in a complex formation process, for example, a carboxyl group, imido and amido groups, and are ligands specifically binding metal ions, for example, ions of zinc, copper, iron, calcium, and magnesium.

An important property of a ligand to form a complex is dissociation constants (pkₙ) of studied compounds in an aqueous solution. The pkₙ values are determined by a potentiometric titration method. The calculation of dissociation constants are performed by the Schwarzenbach method [Grinberg A. A., “An introduction to the chemistry of complex compounds”, fourth ed., Amend., L., Khimiya, 1971].

### pH-Potentiometric Titration Methods

Titration was performed on an apparatus by using a laboratory ionomer I 120.1. A designed galvanic cell consisted of two electrodes, in particular an indicator glass electrode and a silver chloride comparison electrode. The pH value was measured with an accuracy of ±0.2.

Initial 0.05 M solutions of M^{z+} metal ions were prepared from nitrates of zinc (Zn(NO₃)₂.6H₂O), calcium (Ca(NO₃)₂.4H₂O), copper sulfate (CuSO₄.5H₂O), magnesium chloride (MgCl₂.6H₂O) and Mohr's salt ((NH₄)₂Fe (SO₄)₂.6H₂O) . Standardization of the initial solutions of M^{z+} salts was performed by a complexometric titration method with trilon B (M^{z+}═Zn²⁺, Cu²⁺, Mg²⁺, Ca²⁺) and a spectrophotometric method at 490 nm by using o-phenanthroline (M^{z+}═Fe²⁺).

A traditional method for determining acid dissociation step constants (k₁, . . . , kₙ) by using pH-metric titration is the Schwarzenbach method.

A sample of a studied compound (3·10⁻⁴ mol) was dissolved in 15.0 ml of a 0.5M aqueous KNO₃ solution. The obtained 0.2M solution was titrated with a 0.05M aqueous solution of NaOH or HCl under stirring. The amount (V, ml) of the used titrant and the pH value of the solution were registered in each titration point. A titration step was equal to 0.5 ml, and 0.2 ml in the proximity to the equivalency point. Titration was continued until the pH value of the solution reached 11.0 to 11.5. The pH value of the solution corresponding to a=0.5 and/or a=1.5 was determined by the titration curve pH=f(a), and the amount of added gram-equivalents of the titrant was calculated in terms of 1 mole of a titrated compound.

The results of the determination of acid dissociation constants [pkₙ] of the studied compounds by the Schwarzenbach method are given in Table 3.

TABLE 3
pk Values of the studied compounds in an aqueous solution,
calculated by the Schwarzenbach method (C = 0.02M; ionic
strength of a solution = 0.5 (KNO₃); 22° C.)

| Number of a compound | pk₁ | pk₂ |
|---|---|---|
| 2 | 7.11⁽¹⁾ | 11.27⁽²⁾ |
| 3 | 6.88 | 11.27⁽²⁾ |
| 11 | 7.16⁽¹⁾ | 11.89⁽²⁾ |
| 12 | 7.13⁽¹⁾ | 11.95⁽²⁾ |

⁽¹⁾titration with an 0.05M HCl aqueous solution.
⁽²⁾titration with an 0.05M aqueous NaOH solution.

Given the parameters, such as the amount of a studied compound required for potentiometric titration, a testing procedure and a constant calculation method, the Bjerrum method is most suitable and informative [Yu. P. Galaktionov, M. A. Chistyakov, V. G. Sevastiyanov, etc., “Step Stability Constants of Group IIIB Metal and Lanthanide Complexes with Acetylacetone in Aqueous Solution and the Solubility Products of Metal Triacetylacctonates”, Journal of physical chemistry, 2004, v. 78, No. 9, pp. 1596-1604].

The method is based on the view that the process of complex formation is stepwise. A main assumption of the Bjerrum method is an assumption about the formation of only multinuclear complexes. The process of complex formation can be schematically described as follows:

$$\begin{matrix}M + {L}_{↔} ML & {K}_{1} = [ ML ] / [ M ] [ L ] \\ {ML}_{n - 1} + {L}_{↔} {ML}_{n} & {K}_{n} = [ {ML}_{n} ] / [ {ML}_{n - 1} ] [ L ]\end{matrix}$$

According to the Bjerrum method, a studied compound is titrated with a NaOH solution twice: in the absence of and in the presence of a metal ion. The titration is performed under a high constant concentration of an indifferent electrolyte, which provides the solution with a constant ionic strength.

For these purposes, highly soluble salts are used, for example, sodium and potassium salts. Concentration step stability constants (Kₙ) are determined from experiments. In addition, the method provides information concerning the composition of a formed complex, in particular, the maximum possible number of bound ligands (n).

Traditional procedure for determining step stability constants for complexes by the Bjerrum method of pH-metric titration was performed as follows.

A sample of a studies compound (3·10⁻⁴ mol) was dissolved in 14.0 ml of a 0.5M aqueous KNO₃ solution. One ml of a 0.05M aqueous solution of an M^{z+} salt was added under vigorous stirring. The systems were studied with a M^{z+}/L ratio of 1:6. The pH value of a solution was fixed for coexisting ligands and metal ions. The mixture was titrated with a 0.05M NaOH aqueous solution within a broad range of pH values (up to pH 11.0-11.5). The amount of the used titrant (V_{NaoH}, ml) and the pH value of the solution were registered in each titration point. At the moment when a residue formed, the solution became turbid, or the color of the solution changed, the pH value, the nature of the change and the amount of NaOH spent for the titration were registered. The step of titration was 0.25 ml. The value of lgKₙ was determined by the Bjerrum curve n̅=f(pA) for each semi-integer value of the function of complex formation n̅=n−0.5.

Today the Bjerrum method is frequently used to study complex formation processes. This method provides for the determination of two parameters: the concentration of a free ligand under equilibrium conditions ([L⁻]) and the function of complex formation n̅ which is the average number of ligands involved in the complex. Thus, using experimental data, the value [L⁻] can be calculated for each time point of the titration (in particular, −lg[L⁻]=pA), and n̅ can be calculated by equation (1). When a ligand has several dissociating groups, the K_{L} value in equation (1) matches the value of the dissociation constant with the largest pk value.

The Kₙ value is found from the Bjerrum curve (curve of the complex formation) n̅=f(pA) plotted according to equation (2) for each semi-integer value (n̅=n−0.5) of the function of the complex formation. Thus, the step stability constant will be equal to the reciprocal concentration of a free ligand at a point of n̅=n−0.5. Accordingly, the larger the Kₙ value, the more stable a complex compound (chelate) is.

$$\overline{n} = \frac{{c}_{{L}^{-}} - [ {L}^{-} ]}{{c}_{{M}^{z +}}} = \frac{( {10}^{- pH} + {K}_{L} ) \cdot {C}_{NaOH}^{0} \cdot {V}_{NaOH}^{added} - {K}_{L} \cdot {n}_{L}}{{10}^{- pH} \cdot {n}_{{M}^{z +}}} \qquad ( 1 )$$

$${K}_{{st}^{n}} = \frac{1}{[ {L}^{-} ]} \qquad ( 2 )$$

wherein c_{L}⁻ and c_{M}^{z+} are the total concentrations of a ligand and a metal existing in a solution, mol/g;

[L⁻] is the concentration of free ligand ions, mol/g;

K_{L} is the acid dissociation constant of a ligand;

V_{NaOH}^{added} is the volume of a NaOH solution (C_{NaOH}⁰, mol/l) at any time point of titration, added to the titrated mixture of M^{z+} and L, l;

n_{L} and n_{M}^{z+} are the amounts of a ligand compound and a M^{z+} salt in the titrated solution, mol.

The lgK₁ values found by the Bjerrum method for the complexes of the studied compounds with divalent metals are given in Tables 4 and 5. The M^{z+}/L ratio of 1:6 is based on the data concerning the maximum possible coordination number for Zn(II) and Fe(II) (equal to six) and for Cu(II) (equal to four) [Claudia A. Blindauer, M. Tahir Razi, Simon Parsons, Peter J. Sadler, Polyhedron, 2006, v. 25, pp. 513-520].

TABLE 4
The first stability constant of complexes with 1:1
compositions of studied compounds with divalent metal ions
(lgK₁), found by the Bjerrum method (aqueous solution, C_{L} =
0.02M; M^{z+}/L = 1:6; ion strength = 0.5 (KNO₃), 22° C.).

| | The first stability constant of complexes with 1:1 | | | | | |
|---|---|---|---|---|---|---|
| Number | compositions of studied compounds with divalent metal | | | | | |
| of a | ions-lgK₁ | | | | | |
| compound | Zn (II) | Cu (II) | Fe (II) | Fe (III) | Mg (II) | Ca (II) |
| 2 | 4.84 | 4.17 | 5.08 | 5.29 | 2.59 | 2.06 |
| 3 | 5.40 | 5.32 | 5.95 | 6.59 | 4.36 | 4.57 |
| 11 | 5.05 | 4.39 | 6.27 | 6.60 | 2.82 | n̅ < 0.5 |
| 12 | 5.46 | 4.62 | 6.40 | 6.10 | 3.72 | 3.60 |

TABLE 5
The step stability constant of complexes with 1:1
ratio of studied compounds with zinc ions, found by
the Bjerrum method (aqueous solution, C_{L} = 0.02M;
M^{z+}/L = 1:6; ion strength = 0.5 (KNO₃), 22° C.).

| | Step stability constant of complexes with 1:1 ratio of | | |
|---|---|---|---|
| | studied compounds with zinc ions | | |
| Number | The first | The second | The third |
| of a | stability | stability | stability |
| compound | constant, lgK₁ | constant, lgK₂ | constant, lgK₃ |
| 1 | 4.52 | 4.21 | 3.75 |
| 2 | 4.84 | 4.50 | 2.60 |
| 3 | 5.40 | 5.05 | 3.80 |
| 6 | 5.62 | 5.15 | 4.04 |
| 8 | 6.25 | 5.80 | 5.05 |
| 10 | 5.50 | 5.24 | 4.48 |
| 11 | 5.05 | 4.70 | 2.87 |
| 12 | 5.46 | 5.03 | 3.38 |

Thus, the studies have shown that the studied compounds, in particular dicarboxylic acid bisamide derivatives, are effective complex formers, most of which have been found to have high values of lgK₁≥5 for transition metal ions. In addition, the claimed compounds of general formula I have been shown to exhibit a slightly increased ability to form complexes with iron ions.

What is claimed is:

1.-55. (canceled)

56. A dicarboxylic acid bisamide derivative which is:

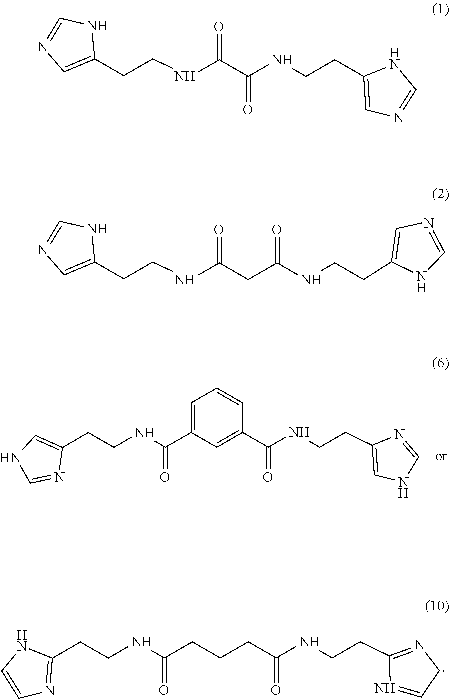 [SMILES: O=C(NCCc1cnc[nH]1)C(=O)NCCc1cnc[nH]1 ; O=C(CC(=O)NCCc1cnc[nH]1)NCCc1cnc[nH]1 ; O=C(NCCc1c[nH]cn1)c1cccc(C(=O)NCCc2c[nH]cn2)c1 ; O=C(CCCC(=O)NCCc1ncc[nH]1)NCCC1=NCC=N1]

57. A pharmaceutical composition comprising the dicarboxylic acid bisamide derivative according to claim 56 or a pharmaceutically acceptable salt thereof, and a pharmaceutically acceptable excipient.

58. A method for the treatment of a disease, comprising administering an effective amount of the pharmaceutical composition of claim 57 to a subject, wherein the disease is selected from an inflammation disease, a viral disease, a cardiovascular disease, a disease associated with oxidation, diabetes and/or its cardiovascular complications, a neurodegenerative disease, a cancer, a disease caused by microbial toxins, alcoholism, alcoholic cirrhosis, iron-excessive anaemia, porphyria cutanea tarda, and transition metal salt poisoning.

59. The method of claim 58, wherein the viral disease is caused by hepatitis C virus, human papilloma virus, HIV, or oncogenic RNA virus.

60. The method of claim 58, wherein the cardiovascular disease is caused by cytostatic cardiotoxicity, cholesterol deposition, or hypertension.
